(19) United States
(12) Patent Application Publication
(10) Pub. No.: US 2003/0138801 A1
(43) Pub. Date: Jul. 24, 2003

(54) Method for the positioning of macromolecules and particles

(76) Inventors: Sven Oscarsson, Uppsala (SE); Arjan Quist, Uppsala (SE); Carl Pahlsson, Uppsala (SE)

Correspondence Address: YOUNG & THOMPSON, 745 SOUTH 23RD STREET 2ND FLOOR, ARLINGTON, VA, 22202

(21) Appl. No.: 10/203,922

(22) PCT Filed: Feb. 16, 2001
(86) PCT No.: PCT/SE01/00355

(30) Foreign Application Priority Data
Feb. 18, 2000 (SE) 0000546-2

(51) Int. Cl. C12Q 1/70; B05D 3/00; C12Q 1/68; C12M 1/34

(52) U.S. Cl. 435/6; 435/287.2; 427/2.11; 435/5

(57) ABSTRACT

The present invention relates to the positioning of nanoparticles on surfaces, and in particular to a method for first positioning nucleic acid polymers onto surface defects on a surface in order to utilise the principle of base pairing for achieving a site specific organisation of particles and macromolecules. The inventive method comprises the positioning of corresponding base pairs in the form of primers on the particles or macromolecules to be positioned, and a high resolution, preferably a resolution of 1-50 nm can be achieved.

The present invention relates to the positioning of nanoparticles on surfaces, and in particular to a method for first positioning nucleic acid polymers on a surface with high precision in order to utilise the principle of base pairing for achieving a site specific organisation of particles and macromolecules. The nucleic acid polymers are positioned using surface defects arranged on the surface using a method wherein said surface defects are created using finely focused ion beam technique or by nano-indenting using a diamond-pointed probe or similar techniques. The inventive method comprises the positioning of corresponding base pairs in the form of primers on the particles or macromolecules to be positioned on the surface. Using this method, a very high resolution can be achieved, e.g. a resolution of 1-50 nm.

### BACKGROUND OF THE INVENTION

Several methods for the immobilisation of particles and macromolecules to surfaces are described in the prior art. Some documents describe methods for arranging molecules on a surface and achieving a location-specific immobilisation with micrometer or nanometer precision. For example Kumar et al., in Acc. Chem. Res., Vol. 28, 1995, pp 219-226, describe a method involving micrometer scale physical indentations on the surface.

Maeda, Y. et al., in J. Vac. Sci. Technol., B 17(2) March/April 1999, pp 494-6, disclose the attachment of streptavidin coated gold particles to DNA templates via biotinylated oligonucleotides. The authors suggest this as an approach to the fabrication of DNA-based nanostructures for a wide range of applications. However, the disclosure of Maeda et al. is based on first attaching the biotinylated probes on the template and lacks a clear strategy for positioning the particles as the DNA itself is not bound to any substrate. Further, a site and type specific binding is not achieved, unless an exact positioning of the DNA can be achieved.

It is also known that DNA can be electrically conducting, and this property has been utilised for so called nanowires in the miniaturisation of electric circuits (M. Ogihara and A. Ray; Nature, vol. 403, Jan. 13, 2000, page 143). This document however fails to disclose how these nanowires would be associated to a substrate, not to mention how this would be done with the necessary high precision.

Niemeyer et al. (Oligonucleotide-directed self-assembly of proteins: semisynthetic DNA-streptavidin hybrid molecules as connectors for the Generation of macroscopic arrays and the construction of supramolecular bioconjugates, Nucl Acids Res, 1994(22), 5530-5539) describes the use of nucleic acid sequences for the positioning of proteins. On page 5536, column 2, Niemeyer et al. admit that methods for the immobilisation of biomolecules on surfaces in a spatially defined way are not yet developed. In the absence of such methods, the results obtained by Niemeyer et al. can not be held to achieve the precision claimed in the cited article.

In PCT/SE98/01712 (WO 99/15895) a method for arraying nanoparticles and individual macromolecules on surfaces through the use of surface defects having a diameter, and a depth and a height, respectively, within the interval of 1-50 nanometers, and a mutual distance within the interval of 0.1-1000 nanometers, the form, appearance and mapping out of said surface defects being adapted to said nanoparticles and/or macromolecules which are to be arrayed. Although a highly effective method, it has a limitation in that only one type of macromolecule is attached to each type of surface defect.

It is remains desirable to be able to perform the immobilisation in as few steps as possible and preferably in one step comprising the simultaneous addition of all particles or molecules to be immobilised. One object of the present invention is therefor to make available a simplified process for positioning or immobilising nanoparticles and/or macromolecules. Another object of the invention is to increase the hitherto available resolution and accuracy in the positioning of nanoparticles and macromolecules.

### SUMMARY OF THE INVENTION

It is the object of the present invention to provide novel artefacts and devices comprising surfaces having particles or macromolecules attached thereon with a high resolution, preferably a resolution in the interval of 1-50 nm, together with methods of their production according to the attached claims, which are hereby incorporated in their entirety.

The advantages of the invention include the possibility of very accurately controlling the positioning of single particles or macromolecules, or types of particles or macromolecules, in two and/or three dimensions and the possibility of accurately influencing the properties and functions of surfaces, for example for creating biomimetic or bioactive surfaces.

Other features and further advantages of the invention will be apparent form the following description and non-limiting examples, and from the attached claims.

### BRIEF DESCRIPTION OF THE DRAWINGS

The present invention will be described in closer detail below, with reference to the examples, claims and drawings, in which

FIG. 1 shows schematically the steps involved in a method, constituting an embodiment of the present invention, and

FIG. 2 shows a digital scan (NanoScope®, Digital Instruments Ltd., scan size 5.000 μm, scan rate 0.9951 Hz, number of samples 256) of a test surface where raised gold dots or peaks have been created on a silica surface (5×5 μm) using a modified electron beam lithograpic technique. The gold dots have a height of about 15 nanometers.

### DESCRIPTION OF THE INVENTION

In the description below, the term “particles or macromolecules” comprises any particle or macromolecule and in particular functional, e.g. bioactive particles or macromolecules, such as enzymes, antibodies, receptor molecules, entire cells or parts thereof, viruses or parts thereof, pharmaceutically active components or their substrates or so called prodrugs.

The term “surfaces” comprises any surface of organic or inorganic origin, irrespective of it being a naturally occurring material or a synthetic carrier. The geometric form or spatial extension, size or shape of the surface can be freely chosen.

The term “surface defects” comprises depressions and/or elevations, such as e.g. holes, indentations, bumps, ridges etc, including elongate surface defects, such as lines, ridges, elongate edges, trenches etc.

The term “resolution” means the degree of definition or accuracy of the achieved two- or three-dimensional pattern of particles or macromolecules on the surface.

The term “nucleic acid polymer” means any synthetic or naturally occurring nucleic acid polymer with a known sequence and preferably a single stranded DNA molecule.

The term “sequence” means the order and number of individual bases in a nucleic acid polymer.

The term “positioning” means the act of making individual particles or macromolecules to become bound to specific locations on a surface or in a three dimensional matrix.

Other terms and expressions used in the description and claims are meant to have the meaning as normally used by a person skilled in the relevant technical field.

The present invention discloses a method, which makes it possible to position or immobilise specific particles or macromolecules with high precision in what basically is a one-step process. In other words, the method allows the simultaneous positioning of a number of different particles or macromolecules in specific and discrete positions, said positions having predetermined x and y co-ordinates or x, y and z co-ordinates. This is possible in the simultaneous presence of all particles or macromolecules to be positioned.

First, surface defects exhibiting very high resolution, preferably having a diameter and a depth and a height, respectively, within the interval of 1-50 nanometers, and a mutual distance within the interval of 0.1-1000 nanometers, the form, appearance and mapping out of said surface defects being adapted to said particles and/or macromolecules which are to be arrayed on the surface.

Alternatively, a substrate having a texture or surface pattern exhibiting very high resolution, preferably a texture or surface pattern with details having a diameter and a depth and a height, respectively, within the interval of 1-50 nanometers, and a mutual distance within the interval of 0.1-1000 nanometers, the form, appearance and mapping out of said details in said texture or pattern being adapted to said particles and/or macromolecules which are to be arrayed on the surface.

The positioning, in its most basic embodiment, is guided by single-stranded or double stranded length of DNA having a known sequence. This DNA is bound to the surface using methods which are described below. The particles or macromolecules of interest are labelled with short nucleic acid sequences or primers, corresponding to specific, shorter sequences on the known sequence bound to the surface. Compared to known methods, the combination of the high resolution physical array described above, and the biospecific or chemical array using the enormous potential of nucleic acid polymers, makes possible a highly accurate and specific arraying with very high resolution and a high degree of control of the end result.

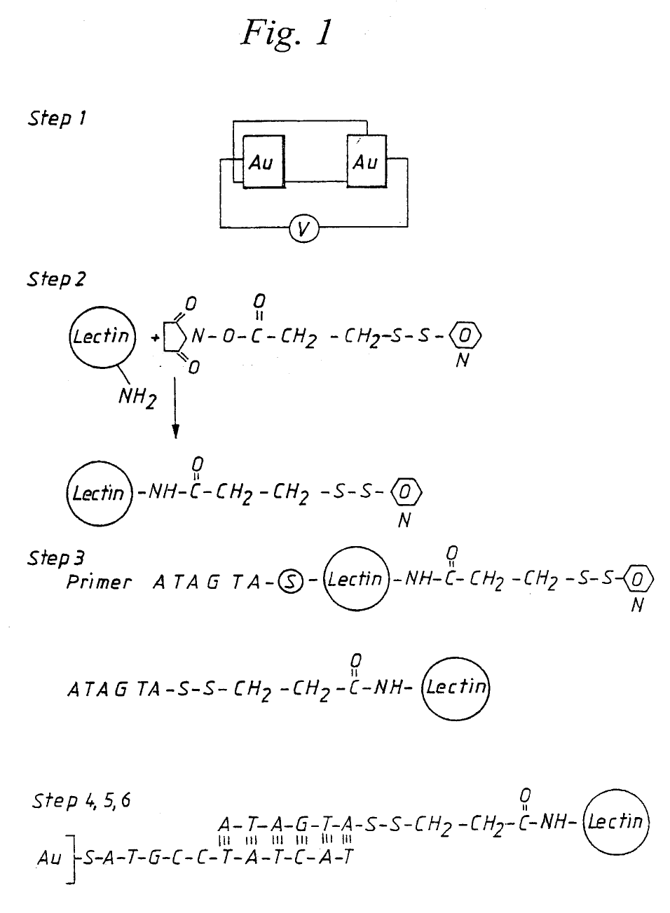

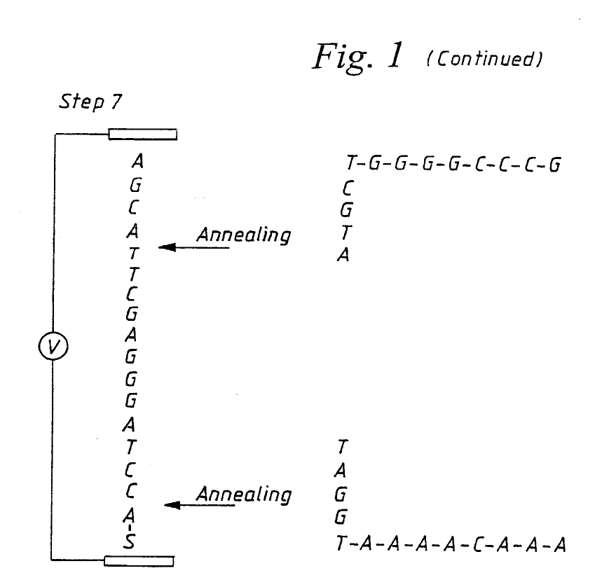

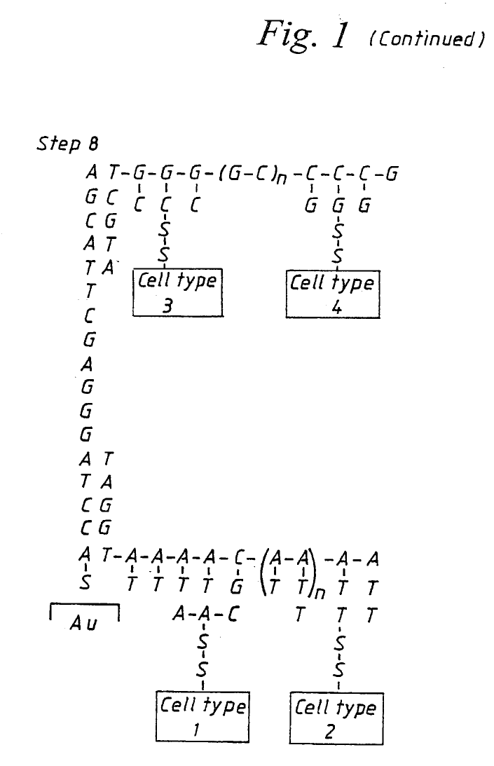

The nucleic acid polymer, which is bound to the surface, can be any one of single stranded DNA, double stranded DNA, RNA and other types of nucleic acid based polymers. There are several techniques for binding these to a surface. One is to connect a base with sulphur in a terminal position to the polymer. Gold can then be applied on two parallel sides of a surface (See FIG. 1, step 1) with the aid of vaporisation or sputtering, using a mask to obtain a site specific deposition of metal. It is known that sulphur binds to gold. It is also known that DNA strands migrate in electric fields. Thus a current applied to the parallel gold surfaces will extend, and straighten the DNA strand. It can then be immobilised using known techniques, such as the MgCl₂ technique, whereby the DNA strand can be permanently bound in an known orientation. Linearization of the nucleic acid polymer as described above is a preferred step, but not entirely indispensable, as it would suffice if the nucleic acid is attached at at least one end.

Alternatively, a base pairing primer with terminal sulphur is used to link the extended DNA strand and attach it to the opposed gold surface and thus locks the DNA in an known orientation.

An alternative to using an electric current is an unilateral flow, which can align the DNA strands if they are covalently bound via the terminal gold. It should be noted that the nucleic acid polymers can be given increased mobility by warming them, leading to maximal repulsion between the charged phosphate moieties. This aids in straightening the polymer strand.

According to the present invention, nucleic acid based strands or polymers with a known sequence and complementary sequences in relation to the bound strand, are synthesised and covalently bound to the particle or macromolecule, or type of particle or macromolecule, which is/are to be positioned at a specific location or locations in an X and Y or X, Y and Z co-ordinate system.

The particles or macromolecules are given specific, complementary primers, directed to the location where the particle or macromolecule is to be immobilised. The particles or macromolecules with their respective nucleic acid fragments or primers are then separated from unbound primers and particles using a suitable method of separation, e.g. desalination using size exclusion chromatography with Sephadex® G25. The mixture is then added to the surface in the presence of detergents which prevent non-specific interactions between the added particles, the surface and nucleic acid polymers, already immobilised to the surface. The labelled particles or macromolecules are immobilised to their predetermined locations through selective base pairing reactions.

Although it is known that DNA can be electrically conducting, it is surprising and forms part of the present invention that the nucleic strands used for positioning also can be used for sending or receiving signals in the form of electric current, to and from the immobilised particles or macromolecules. Thus, the nucleic strands can be used to regulate e.g. the activity, the conformation or charge of particles or macromolecules. Conversely, they can be used to transmit a signal, emanating from a molecule or particle as a result of said molecule or particle taking part in a chemical or physical reaction. The conducting nucleic acid polymers can make possible the use of the particles or macromolecules as regulators, sensors etc. This makes it possible to produce extremely sensitive sensors and gives the possibility to regulate for example biomimetic surfaces and thus to create artificial sensors and activators for incorporation in a living being.

One embodiment of the invention comprises biomimetic surfaces, their construction and control thereof. Examples of biomimetic surfaces include biocatalytic surfaces, for example biocatalysts for performing analyses and/or syntheses. Biocatalytic surfaces are also suitable for medical use, for example intra or exo corporeal production or elimination of biologically active components, such as signal substances, products of metabolic malfunctions etc.

Further, the inventive surfaces can be used for avoiding macromolecular adsorption or unwanted binding, e.g. fouling, in medical and analytical applications.

According to one embodiment of the invention, a gradient with respect to a chemical or physical property is created by attaching nucleic acid polymers having known sequences to a surface. The particles or macromolecules to be positioned are labelled with known nucleotide sequences in the form of primers, corresponding to specific locations on the nucleic acid polymers attached to the surface, and the labelled particles or macromolecules are then brought in contact with the surface bound nucleic acid polymers under conditions allowing binding between corresponding base pairs. Suitable chemical or physical properties are specific binding or affinity properties, such as naturally occurring or synthetic receptor molecules, binding proteins, immunologically active cells or receptors, pH, electric charge etc.

The above gradient can be adapted to become a three dimensional gradient, either by constructing a three dimensional nucleic acid polymer matrix on the surface, or by adding further particles or macromolecules as a secondary structure on the primary structure of polymers and labelled particles.

The above gradients, either two or three dimensional, can be used for separation and/or detection of macromolecules, for creation of a mixed separation surface where the upper part of the surface is a hydrophobic surface and the lower part is an ionic surface which can be created by introduction of ion exchangeable groups via the DNA matrix or hydrophobic clusters of macromolecules for hydrophobic chromatography. Other possibilities are to create a gradually increasing hydrophobicity of the surface from a hydrophilic surface via an amphiphilic character to a pure hydrophobic surface in the same separation unit. Concerning applications in the area of microbiology the possibility exist to produce a mosaic or pattern of different kind of bacteria on predetermined positions. This is applicable in food industry, for example to regulate pH by acetic acid producing bacteria or other interesting products for regulation of micro- or even nano-environments.

According to another embodiment of the present invention, the method can be used for creating novel bio-compatible materials or to form an interface between an artificial implant and a living organism. The inventive method can thus be used for creating a surface on an implant, which surface eliminates or minimises autoimmune reactions or rejection mechanisms, or which surface enhances the incorporation of the implant by the surrounding tissue. Examples of implants and artefacts, suitable for the inventive method include dental implants, surgical implants such as stents, artificial joints and neurological implants, such as sleeves for repairing damaged nerves, the interface between a prosthetic device and the original muscles and/or nerves etc.

A surface according to the invention can function as an interface between electric circuits and the nervous system of an animal, such as circuits delivering impulses to the brain in connection with seeing and hearing aids, or sensors and circuits for the control of prosthetic limbs.

A further embodiment of the present invention concerns the production of biomolecular memories and circuits, capable of storing, retrieving and processing information.

Another embodiment of the present invention concerns drug delivery, where a surface with immobilised particles or macromolecules can be used as a depot of a pharmaceutically active substance or a precursor of such substance. The possibility of incorporation bioactive molecules and the conductive properties of the nucleic acid polymers used for immobilising the particles or macromolecules opens up the possibilities of construction of a nanoscale, intra corporeal device for active release of a drug, for example triggered by a change in the surrounding environment. Such a change, detectable by one or several particles or macromolecules immobilised on the surface, can be a change in the concentration of a metabolite, a signal substance, a hormone or a mediator, associated with the medical condition to be treated.

Further embodiments of the present invention can be found in the field of sensors. By using the inventive method to immobilise specific particles or macromolecules for example to piezoelectric crystals, a sensor is created which detects the adsorption of molecules to the coated crystal surface as a change in frequency of the crystal. Such sensors can be used in analytical and medical applications, for example for detecting the presence of trace amounts of specific compounds.

The present invention also gives the possibility of tailoring unique surfaces, giving them specific properties with regards to chemical or physical properties, such as friction, conductivity, reflectance, specific binding or repelling properties etc. Such surfaces and artefacts with surfaces according to the present invention have utility in medicine, electronics, micromechanics, analysis and synthesis etc.

### EXAMPLE

### Example 1

The following non-limiting example refers to FIG. 1, attached to the description.

On a silicon substrate, a mask having a width of 1 μm is arranged. Gold is sputtered onto the surface, whereupon the mask is removed. Thereby a gap of 1 μm is formed between two parallel gold surfaces, which are used as electrodes for linearising or “stretching” nucleic acid molecules over the gap. A potential of 1 kV/cm is applied over the electrodes. (Step 1)

Lectin from Simbucus nigra (1 mg/ml in 0.1 phosphate buffer, pH 7.5) is mixed with SPDP (from a stock solution of about 50 mM SPDP in EtOH) to a final concentration of 10 mM SPDP. Following a reaction time of 15 minutes, during which the progress of the reaction is studied at 343 nm, the reaction is terminated through desalination using a PD 10 chromatography column. Thereby surplus reactants and by-products are removed from the lectin, having ssPy groups. (Step 2)

Primers (1 ml) having terminal base pairs containing sulphur at one end (MW 6500, concentration 5·10⁻⁶ M) and having complementary bases (to a single stranded DNA, ssDNA) at the other end, are then added to the disulphide containing lectin in a concentration of 5·10⁻⁷ M. A covalent bond is formed between the lectin and the primer within 15 minutes at room temperature in a phosphate buffer, pH 7.5. The progress of the reaction is again observed at 343 nm and surplus primers are then removed from the formed conjugate using a PD 10 column. (Step 3)

ssDNA with terminal sulphur is added to the gap between the electrodes. The reaction between terminal sulphur and the gold electrodes takes place within about 30 minutes. A potential is created over the electrodes in order to align the molecules. The single stranded DNA molecules will move towards the positive electrode, but as the terminal sulphur is covalently bound to the other gold electrode, the polymer will be aligned between the electrodes. The DNA is fixed in the straight position by the addition of 1 mM Mg solution to the gap. (Step 4)

Then the lectin having a modified primer is added and after 15 minutes at 40 degrees C, base pairing between the complementary parts will have taken place. (Step 5)

The lectin is thus positioned at predetermined positions on the surface, with the aid of the ssDNA functioning as a co-ordinate axis. (Step 6)

More advanced patterns can be created using further ssDNA or other nucleic acid molecules having site specific complementary terminal base pairs in relation to the primary structure of nucleic acid polymers, immobilised to the surface. In this manner a 3-dimensional pattern can be created. After annealing the second group of polymers, an electric potential can be applied. aligning the second polymers in a direction different to the direction of the first polymers, immobilised to the surface. (Step 7) Following this, different macromolecules or particles, e.g. cells can be attached to the nucleic acid polymer frame work. (Step 8)

### Example 2

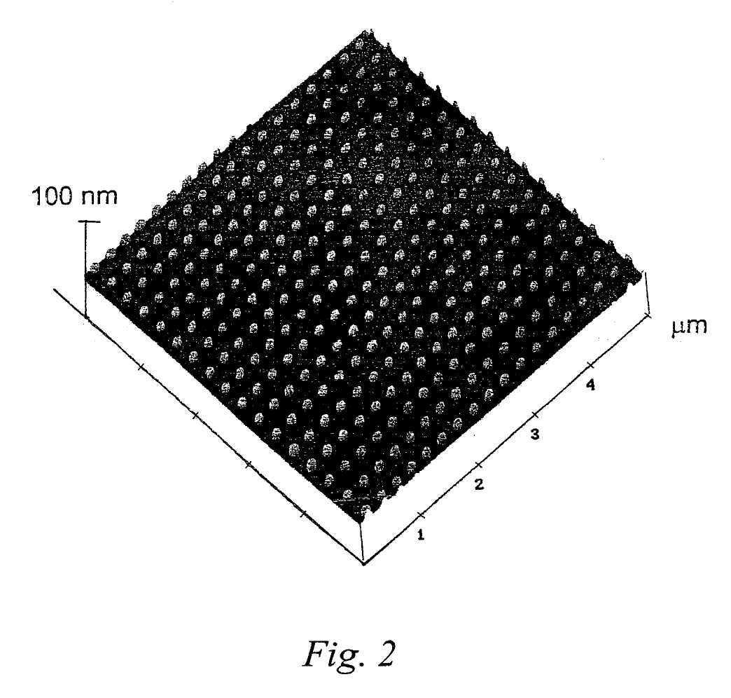

The following non-limiting example refers to FIG. 2, attached to the description.

Using electron beam lithography, discrete dots of gold were created on a silicon wafer. The electron beam lithography was performed according to the following procedure: PMMA was applied evenly to the silicon wafer by spinning and drying the wafer. An electron beam, guided with high precision, was used to damage the PMMA layer at predetermined positions, forming a pattern on the surface of the wafer. Gold was vaporised over the wafer and attaches firmly at the locations where the silicon is exposed. Surplus gold was removed by a solvent treatment, removing the PMMA layer and gold.

The dots were located at a distance of each other of 1 μm and their height was determined to be about 15 nm (NanoScope®, Digital Instruments Ltd., scan size 5.000 μm, scan rate 0.9951 Hz, number of samples 256).

These dots would then be used for positioning nucleic acid polymers, which nucleic acid polymers in turn would be used to position macromolecules using specific complementary base pairs in relation to the structure of the immobilised nucleic acid polymer, according to the present invention.

Although the invention has been described with regard to its preferred embodiments, which constitute the best mode presently known to the inventors, it should be understood that various changes and modifications as would be obvious to one having the ordinary skill in this art may be made without departing from the scope of the invention as set forth in the claims appended hereto.

1. A method for the positioning of particles or macromolecules on a surface, characterized in that
surface defects having a diameter, a depth, and a height, respectively, within the interval of 1-50 nanometers, and a mutual distance within the interval of 0.1-1000 nanometers, the form, appearance and mapping out of said surface defects being adapted to said particles or macromolecules which are to be arrayed, are arranged on said surface,
nucleic acid polymers having known sequences are attached to said surface defects,
the particles or macromolecules to be positioned are labelled with known nucleotide sequences in the form of primers, corresponding to specific locations on the nucleic acid polymers attached to the surface, and
the labelled particles or macromolecules are brought in contact with the surface bound nucleic acid polymers under conditions allowing binding between corresponding base pairs.

2. The method according to claim 1, characterized in that the nucleic acid molecules are single stranded DNA molecules.

3. The method according to claim 1, characterized in that the particles or macromolecules are chosen among bioactive particles or macromolecules.

4. The method according to claim 3, characterized in that the particles or macromolecules are chosen among enzymes, hormones, signal substances, antibodies, and receptor molecules, cells or parts thereof, bacteria, viruses or parts thereof.

5. The method according to claim 1, characterized in that the particles or macromolecules are chosen among pharmaceutically active substances.

6. A two dimensional gradient with respect to chemical or physical properties, characterized in that said gradient
consists of particles or macromolecules bound to a surface via nucleotide sequences binding to corresponding sequences attached to the surface, and
exhibits a resolution of 1-50 nanometers.

7. A three dimensional gradient with respect to chemical or physical properties, characterized in that said gradient
consists of primary particles or macromolecules bound to a surface via nucleotide sequences binding to corresponding sequences attached to the surface, and
secondary particles or macromolecules, bound the said primary particles, and
exhibits a resolution of 1-50 nanometers in at least one dimension.

8. Use of a gradient according to any one of claims 6-7 for the separation of macromolecules.

9. Use of a gradient according to any one of claims 6-7 for the separation of cells.

10. Use of a gradient according to any one of claims 6-7 for the separation of bacteria or viruses.

11. An array of particles and/or macromolecules immobilised to a surface obtained by the method according to any one of claims 1-5.

12. A two-dimensional array of particles and/or macromolecules immobilised to a surface, characterized in that the particles and/or macromolecules are bound to the surface through nucleic acid sequences, bound to said particles and/or macromolecules, having sequences complementary to sequences on nucleic acid polymers, bound to the surface.

13. A three-dimensional array of particles and/or macromolecules immobilised to a surface, characterized in that a first layer of particles and/or macromolecules are bound to the surface through nucleic acid sequences, bound to said particles and/or macromolecules, having sequences complementary to sequences on nucleic acid polymers, bound to the surface, and a second and further layers of particles and/or macromolecules are bound to said first layer.

14. An array according to any one of claims 11-13, characterized in that the conductivity of the nucleic acid sequences is utilised for the transmission of signals from or to the bound particles and/or macromolecules.

15. An array according to any one of claims 11-13, characterized in that the surface is a piezoelectric material.

16. A device for incorporation in an animal or human body, characterized in that the at least one surface of said device is covered with particles and/or macromolecules bound to a surface via nucleotide sequences binding to corresponding sequences attached to said surface.

17. A device for controlled drug release, characterized in that said device incorporates pharmaceutically active substances or components of such substances in the form of particles and/or macromolecules bound to a surface via nucleotide sequences binding to corresponding sequences attached to said surface.

18. A device constituting an interface between the nerve paths of a living being and the electronic circuitry of an prosthetic device, characterized in that said device comprises a surface having particles and/or macromolecules bound to said surface via nucleotide sequences binding to corresponding sequences attached to said surface.

19. A device capable of storing, retrieving and/or processing information, characterized in that said device comprises a surface having particles and/or macromolecules bound to said surface via nucleotide sequences binding to corresponding sequences attached to said surface.

20. A sensor for detecting the presence of a given entity, characterized in that said sensor comprises a surface having particles and/or macromolecules which the capability of reacting with, binding to or otherwise changing their chemical or physical state in the presence of said entity, said particles and/or macromolecules being bound to the surface via nucleotide sequences binding to corresponding sequences attached to the surface.

21. A sensor according to claim 20, characterized in that the surface is the surface of a piezoelectric crystal.
